(19) United States
(12) Patent Application Publication
(10) Pub. No.: US 2015/0173629 A1
(43) Pub. Date: Jun. 25, 2015

(54) DEVICE, SYSTEM, AND METHOD FOR ASSESSING INTRAVASCULAR PRESSURE

(71) Applicant: Volcano Corporation, San Diego, CA (US)

(72) Inventors: Paul Douglas Corl, Palo Alto, CA (US); Mark Richardson, Escondido, CA (US); Andrew Tochterman, Carlsbad, CA (US); John Unser, Temecula, CA (US); Curtiss Stinis, San Diego, CA (US)

(21) Appl. No.: 14/573,734

(22) Filed: Dec. 17, 2014

Related U.S. Application Data
(60) Provisional application No. 61/918,601, filed on Dec. 19, 2013.

(51) Int. Cl. A61B 5/0215; A61B 6/12; A61B 19/00; A61B 5/02

(52) U.S. Cl. CPC A61B 5/02158; A61B 5/02007; A61B 6/12; A61B 19/54; A61B 2019/5466

(57) ABSTRACT

What is described is an apparatus for intravascular pressure measurement, comprising an elongate body and a first pressure sensor. The elongate body includes a proximal portion and a distal portion, the body defines a lumen extending from a proximal end to a distal end of the body, the lumen is sized and shaped to allow the passage of a guidewire therethrough, and the body includes an annular wall extending from the lumen to an outer surface of the body. The first pressure sensor is disposed entirely within the wall of the distal portion of the body, and the pressure sensor includes a sensor cover coupled to the wall. An exterior surface of the sensor cover and the outer surface of the body are substantially aligned.

### CROSS REFERENCE TO RELATED APPLICATIONS

The present application claims priority to and the benefit of the U.S. Provisional Patent Application No. 61/918,601, filed Dec. 19, 2013, which is hereby incorporated by reference in its entirety.

### TECHNICAL FIELD

Embodiments of the present disclosure relate generally to the field of medical devices and, more particularly, to a device, system, and method for assessing pressure within vessels. In particular, the present disclosure relates to the assessment of the severity of a blockage or other restriction to the flow of fluid through a vessel. Aspects of the present disclosure are particularly suited for evaluation of biological vessels in some instances. For example, some particular embodiments of the present disclosure are specifically configured for the evaluation of a stenosis of a human blood vessel.

### BACKGROUND

Heart disease is a serious health condition affecting millions of people worldwide. One major cause of heart disease is the presence of blockages or lesions within the blood vessels that reduce blood flow through the vessels. Traditionally, interventional cardiologists have relied on X-ray fluoroscopic images with injection of X-ray contrast medium into the artery of interest to highlight the silhouette of the vessel lumen to guide treatment. Unfortunately, the limited resolution and discrete projections provided by X-ray fluoroscopy often yield insufficient information to accurately assess the functional significance (i.e., impairment of blood flow) attributable to an obstruction.

Improved techniques for assessing the functional significance and likely benefit of treatment of a stenosis in a blood vessel are the calculation of fractional flow reserve (FFR) and instantaneous wave-free ratios. FFR is defined as the ratio of the maximal hyperemic blood flow in a stenotic artery compared to what the maximal flow would be if the stenosis were alleviated. Instantaneous wave-free ratio is defined as the ratio of blood flow in a stenotic artery distal to the stenosis during the wave-free period of diastole compared to the aortic pressure. Both FFR and the instantenous wave-free ratio values are calculated as the ratio of the distal (to the stenosis) pressure to the proximal (typically aortic) pressure, sometimes also including a small correction to account for the effect of the venous pressure. Both FFR and the instantaneous wave-free ratio provide an index of stenosis severity that allow determination if the obstruction limits blood flow within the vessel to an extent that intervention is warranted, taking into consideration both the risks and benefits of treatment. The more restrictive the stenosis, the greater the pressure drop across the stenosis, and the lower the resulting FFR or instantaneous wave-free ratio. Both FFR and instantaneous wave-free ratio measurements can be used to establish a criterion for guiding treatment decisions. The ratio in a healthy vessel is by definition 1.00. FFR values less than about 0.80 are generally deemed to indicate a functionally significant lesion likely to benefit from treatment, while values above 0.80 indicate reduced likelihood of net benefit from intervention. Instantaneous wave-free ratio values have been correlated to FFR values whereby a value of 0.89 approximates an FFR of 0.80. Common treatment options include angioplasty or atherectomy with stent implantation, or surgical bypass of the obstructed artery.

One method for measuring the proximal and distal pressures used for FFR calculation is to advance a pressure sensing guidewire (with a pressure sensor embedded near its distal tip) across the lesion to a distal location, while the guiding catheter (with an attached pressure transducer) is used to provide a pressure measurement proximal to the stenosis (typically in the aorta or the ostium of the coronary artery). Despite the level of evidence in the guidelines, the use of pressure sensing guide wires remains relatively low (estimated less than 6% of cases worldwide). The reasons are partially tied to the performance of the pressure guide wires relative to that of standard angioplasty wires. Incorporating a pressure sensor into a guidewire generally requires compromises in the mechanical performance of the guidewire in terms of steerability, durability, stiffness profile, etc., that make it more difficult to navigate the coronary circulation to deliver the guidewire or subsequent interventional catheters across the lesion. As such, physicians will often abandon use of a pressure sensing guidewire when they experience challenges steering the pressure guide wire distal to the disease. And it is common where a physician may not even try a pressure guide wire, despite a desire to do so, because the anatomy appears visually as too challenging. Efforts continue to design pressure guide wires to perform more like standard angioplasty wires, but there are inherent design limitations that prevent that from happening.

Another method of measuring the pressure gradient across a lesion is to use a small catheter connected to an external blood pressure transducer to measure the pressure at the tip of the catheter through a fluid column within the catheter, similarly to the guiding catheter pressure measurement. However, this method can introduce error into the FFR calculation because as the catheter crosses the lesion, it creates additional obstruction to blood flow across the stenosis and contributes to a lower distal blood pressure measurement than what would be caused by the lesion alone, exaggerating the apparent functional significance of the lesion.

FIGS. 1 and 2 illustrate this phenomenon. FIG. 1 shows computer-derived calculations indicating the overestimation of the pressure gradient across a 10 mm long stenotic lesion in the presence of a 0.015″ and a 0.018″ guidewire for varying area stenosis and reference diameters at a flow rate of 1 mL/s. FIG. 2 shows computer-derived calculations indicating the overestimation of the pressure gradient across a stenotic lesion in the presence of a 0.015 in guidewire for varying areas of stenosis at two different flow rates. As shown, the percent overestimation of the pressure gradient due to the presence of a wire through the stenosis increased dramatically with the severity of the stenosis and decreased with the increasing reference diameter of the diseased vessel. Moreover, the graphs imply that in small coronary arteries the overestimation of the measured pressure gradient by the presence of the wire itself will be larger than in large coronary vessels for a given percent stenosis. FIGS. 1 and 2 are sourced from “Coronary Pressure From a Physiological Index,” by B. D. BeBruyne (Thesis at Catholic University of Lourain Medical School, 1995, pp. 46-47). Thus, both pressure-sensing guidewires and pressure-sensing catheters may give exaggerated pressure gradient measurements across a lesion.

While the existing treatments have been generally adequate for their intended purposes, they have not been entirely satisfactory in all respects. The devices, systems, and associated methods of the present disclosure overcome one or more of the shortcomings of the prior art.

### SUMMARY

In one exemplary embodiment, the present disclosure describes an apparatus for intravascular pressure measurement, comprising: an elongate body including a proximal portion and a distal portion, the body defining a lumen extending from a proximal end to a distal end of the body, the lumen sized and shaped to allow the passage of a guidewire there through, the body including an annular wall extending from the lumen to an outer surface of the body; and a first pressure sensor disposed within the wall of the distal portion of the body, the pressure sensor including a sensor cover coupled to the wall, wherein an exterior surface of the sensor cover and the outer surface of the body are substantially aligned. The apparatus can include at least one perfusion port in the wall that enables fluid communication between the lumen and environmental contents outside the elongate body. The at least one perfusion port can include an aperture extending through the wall from the outer surface of the body to the lumen.

In another exemplary embodiment, the present disclosure describes a method for intravascular pressure measurement within a lumen of a vessel including a lesion, comprising: positioning a guidewire within the lumen of the vessel distal to the lesion; advancing a pressure-sensing catheter including a first pressure sensor and at least one perfusion port over the guidewire within the lumen of the vessel such that the first pressure sensor is positioned distal to the lesion; withdrawing the guidewire in a proximal direction until the guidewire is positioned proximal of the at least one perfusion port; and obtaining a distal pressure measurement from the first pressure sensor. The method can also include imaging the pressure-sensing catheter to obtain image data reflecting the location of the first pressure sensor within the lumen relative to the lesion and repositioning the pressure-sensing catheter in an optimal intravascular location for pressure measurement based on the image data. The method can also include withdrawing the pressure-sensing catheter in a proximal direction to position the first pressure sensor proximal to the lesion, withdrawing the guidewire in a proximal direction until the guidewire is positioned proximal of both the lesion and the at least one perfusion port, and obtaining a proximal pressure measurement from the first pressure sensor.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a graph illustrating a computer-derived calculation of the overestimation of a pressure gradient across a stenotic lesion in the presence of two different guidewires.

FIG. 2 is a graph illustrating a computer-derived calculation of the overestimation of a pressure gradient across a stenotic lesion having varying stenotic areas in the presence of a guidewire at two different flow rates.

FIG. 3 is a block diagram of a medical system including a side view of an exemplary pressure-sensing catheter according to one embodiment of the present disclosure.

FIG. 4 is a perspective view of a distal portion of an exemplary pressure-sensing catheter having an over-the-wire configuration according to one embodiment of the present disclosure.

FIG. 5 is a cross-sectional side view of a portion of the pressure-sensing catheter shown in FIG. 4 that includes an exemplary pressure sensor.

FIG. 6 is a perspective view of a distal portion of an exemplary pressure-sensing catheter having a rapid exchange configuration according to one embodiment of the present disclosure.

FIG. 7 is a partially cross-sectional view of an exemplary pressure-sensing catheter according to one embodiment of the present disclosure.

FIG. 8 is a partially cross-sectional view of an exemplary pressure-sensing catheter including an exemplary perfusion port according to one embodiment of the present disclosure.

FIG. 9 is a partially cross-sectional view of an exemplary pressure-sensing catheter having a rapid exchange configuration according to one embodiment of the present disclosure.

FIG. 10 is a partially cross-sectional view of an exemplary pressure-sensing catheter including an exemplary perfusion port and having a rapid exchange configuration according to one embodiment of the present disclosure.

FIG. 11 is a partially cross-sectional view of an exemplary pressure-sensing catheter including multiple pressure sensors according to one embodiment of the present disclosure.

FIG. 12 is a partially cross-sectional view of an exemplary pressure-sensing catheter including multiple pressure sensors and multiple exemplary perfusion ports according to one embodiment of the present disclosure.

FIG. 13 is a partially cross-sectional view of an exemplary pressure-sensing catheter including multiple pressure sensors and having a rapid exchange configuration according to one embodiment of the present disclosure.

FIG. 14 is a partially cross-sectional view of an exemplary pressure-sensing catheter including multiple pressure sensors and multiple exemplary perfusion ports and having a rapid exchange configuration according to one embodiment of the present disclosure.

FIGS. 15A and 15B illustrate a method of using an exemplary pressure-sensing catheter having a single pressure sensor and an exemplary perfusion port positioned within a diseased vessel to measure a distal pressure.

FIGS. 16A and 16B illustrate a method of using the exemplary pressure-sensing catheter shown in FIG. 15A to measure a proximal pressure within the diseased vessel.

FIGS. 17A and 17B illustrate a method of using an exemplary pressure-sensing catheter having multiple pressure sensors and multiple exemplary perfusion ports positioned within the diseased vessel to measure both proximal and distal pressures.

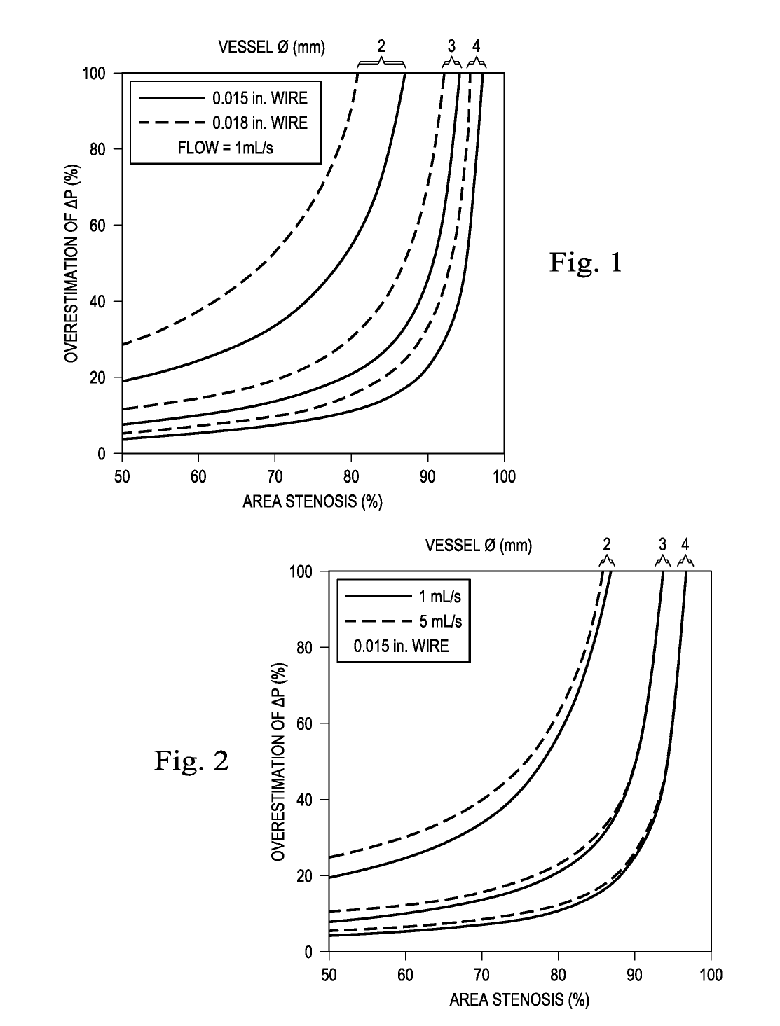

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. For the sake of brevity, however, the numerous iterations of these combinations will not be described separately. For simplicity, in some instances the same reference numbers are used throughout the drawings to refer to the same or like parts.

The present disclosure relates generally to a device, systems, and methods of using a pressure-sensing catheter for the assessment of intravascular pressure, including, by way of non-limiting example, the calculation of an FFR value. These measurements can also be made in the peripheral vasculature including but not limited to the Superficial femoral artery (SFA), below the knee (BTK, i.e. Tibial), and Iliac artery. In some instances, embodiments of the present disclosure are configured to measure the pressure proximal to and distal to a stenotic lesion within a blood vessel. Embodiments of the present disclosure include a pressure sensor embedded in the wall of the catheter instead of being encased in a bulky housing attached to the catheter. In some embodiments, the pressure-sensing catheter disclosed herein includes at least one perfusion port extending through the catheter wall to allow for blood flow through the catheter lumen. In some embodiments, the pressure-sensing catheter disclosed herein is configured as a rapid exchange catheter. In other embodiments, the pressure-sensing catheter disclosed herein is configured as a conventional over-the-wire catheter. The pressure-sensing catheters disclosed herein enable the user to obtain pressure measurements using an existing guidewire (e.g., a conventional 0.014 inch guidewire) that can remain fairly stationary through the pressure measurement procedure. Thus, the pressure-sensing catheters disclosed herein enable the user to obtain physiologic information about an intravascular lesion upon pullback of the catheter without losing the original position of the guidewire.

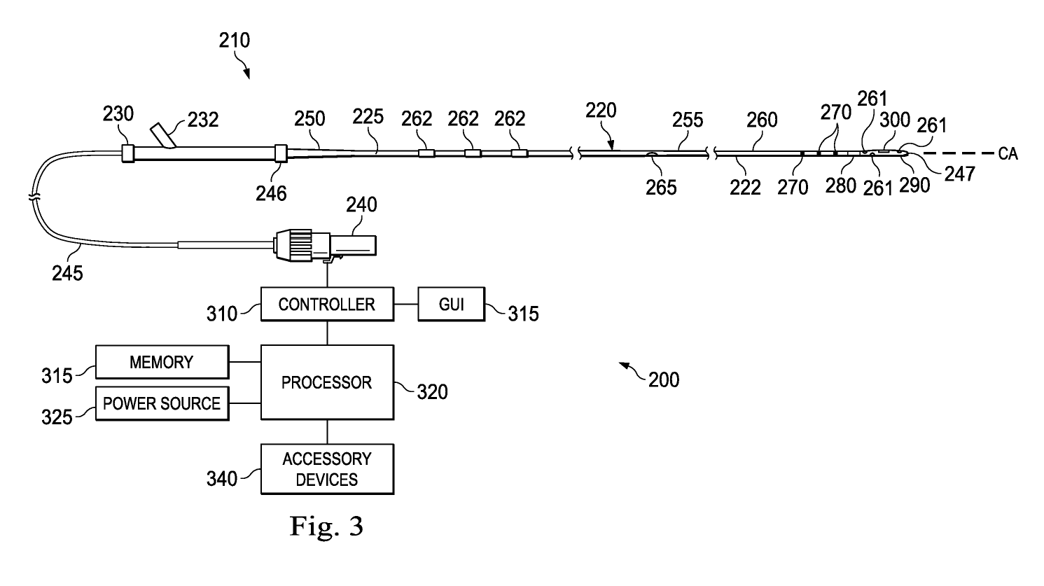

FIG. 3 illustrates a medical system 200 that is configured to measure pressure within a tubular structure (e.g., a blood vessel) according to one embodiment of the present disclosure. In some embodiments, the medical system 200 is configured to calculate a pressure ratio (i.e. FFR) based on the obtained pressure measurements. The system 200 includes a pressure-sensing catheter 210 comprising an elongate, flexible, tubular body 220. The body 220 comprises a catheter wall 222 that defines an internal lumen 225. In general, the body 220 is sized and shaped for use within an internal structure of a patient, including but not limited to a patient's arteries, veins, heart chambers, neurovascular structures, gastrointestinal system, pulmonary system, and/or other areas where internal access of patient anatomy is desirable. In the pictured embodiment, the body 220 is shaped and sized for intravascular placement.

In particular, the body 220 is shaped and configured for insertion into a lumen of a blood vessel (not shown) such that a longitudinal axis CA of the catheter 100 aligns with a longitudinal axis of the vessel at any given position within the vessel lumen. In that regard, the straight configuration illustrated in FIG. 3 is for exemplary purposes only and in no way limits the manner in which the catheter 200 may curve in other instances. Generally, the elongate body 220 may be configured to take on any desired arcuate profile when in the curved configuration. The body 220 is formed of a flexible material such as, by way of non-limiting example, plastics, high density polyethylene, polytetrafluoroethylene (PTFE), Nylon, block copolymers of polyamide and polyether (e.g., PEBAX), thermoplastic, polyimide, silicone, elastomers, metals, shape memory alloys, polyolefin, polyether-ester copolymer, polyurethane, polyvinyl chloride, combinations thereof, or any other suitable material for the manufacture of flexible, elongate catheters.

The body 220 extends from an adapter 230 along the longitudinal axis CA. In the pictured embodiment, the body 220 is integrally coupled to the adapter 230. In other embodiments, the body 220 may be detachably coupled to the adapter 230, thereby permitting the body 220 to be replaceable. The adapter 230 is configured to couple the catheter 200 to another medical device through a port 232 and/or an electrical connection 245. The port 232 may be configured to receive fluid there through, thereby permitting the user to irrigate or flush the lumen 225. Various medical devices that may be coupled to the catheter 200 include, by way of non-limiting example, a storage vessel, a disposal vessel, a vacuum system, a syringe, an infusion pump, and/or an insufflation device. For example, the port 232 may include a Luer-type connector capable of sealably engaging an irrigation device such as a syringe. Various devices that may be coupled to the catheter 200 by the electrical connection 245 include, by way of non-limiting example, an energy generator (e.g., an ultrasound generator), a power source, a patient interface module (“PIM”), a computer system, and/or a surgical console. In the pictured embodiment, the adapter 230 couples the body 220 to an interface 240 by the electrical connection 245.

The body 220 includes a proximal portion 250, and intermediate portion 255, and a distal portion 260. The proximal portion 250 of the body 220 connects to the adapter 230, which may be sized and configured to be held and manipulated by a user outside a patient's body. By manipulating the adapter 230 outside the patient's body, the user may advance the body 220 of the catheter 210 through an intravascular path and remotely manipulate or actuate the distal portion 260 holding the sensor 300. The lumen 225 allows for the passage of contents from the distal portion 260 to the proximal portion 250, and in some instances through the adapter 230. The lumen 225 is shaped and configured to allow the passage of fluid, cellular material, or another medical device from a proximal end 246 to a distal end 247 (and/or a guidewire port 265). In some embodiments, the lumen 225 is sized to accommodate the passage of a guidewire. In such an embodiment, the lumen 225 has an internal diameter greater than 0.014 inches. In some embodiments, the body 220 includes more than one lumen.

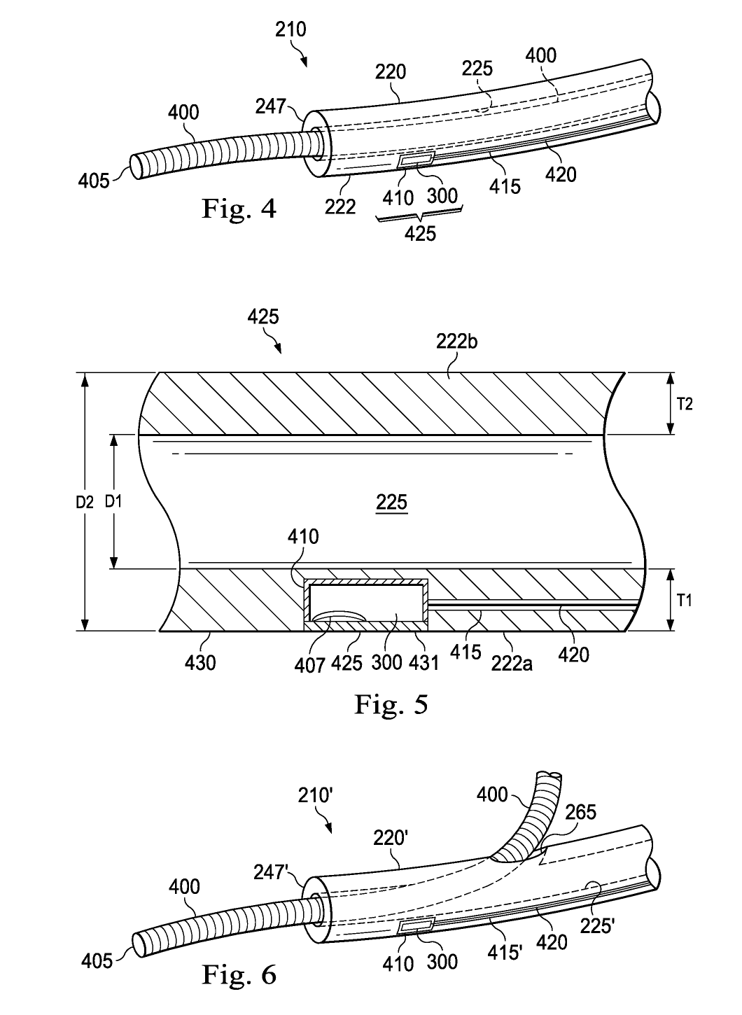

In FIG. 3, the catheter 210 includes multiple perfusion ports 261. The perfusion ports are disposed at the distal portion 260 of the catheter 210. The perfusion ports 261 extend through the body 220 to permit fluid exchange between the lumen 225 and the environment outside the distal portion 260 of the catheter 210. Other embodiments may lack the perfusion ports 261. The perfusion ports 261 will be described further below in relation to FIGS. 4-6.

In the pictured embodiment, the proximal portion 250 of the catheter 210 includes shaft markers 262 to aid in positioning the catheter 210 in the body of a patient. The shaft markers 262 may be visible to the naked eye. In some embodiments, the shaft markers 262 may indicate the relevant insertion distance from a particular anatomical entry point, such as the radial artery and/or the femoral artery.

The intermediate portion 255 may include the guidewire port 265 from which a guidewire may enter or emerge. In other embodiments, the guidewire port 265 may be disposed elsewhere on the catheter 210. Other embodiments may lack a guidewire port 265. The guidewire port 265 may be formed at a variety of distances along the elongated body 220. In some embodiments the distance between the guidewire port 265 and the distal end 247 ranges from about 10 cm to about 20 cm. For example, in one embodiment the distance between the guidewire port 265 and the distal end 247 ranges from about 10 cm to about 12 cm. These examples are provided for illustrative purposes only, and are not intended to be limiting.

In the pictured embodiment, the distal portion 260 includes several radiopaque markers 270. Each radiopaque marker 270 may be coupled to the catheter wall 222 at a known distance from the pressure sensor 300 and/or the distal end 247. The radiopaque markers 270 permit the physician to fluoroscopically visualize the location and orientation of the markers, the distal end 247, and the pressure sensor 300 within the patient. For example, when the distal portion 260 extends into a blood vessel in the vicinity of a lesion, X-ray imaging of the radiopaque markers 270 may confirm successful positioning of the pressure sensor 300 distal to or proximal to the lesion. In some embodiments, the radiopaque markers 270 may circumferentially surround the body 220. In other embodiments, the radiopaque markers 270 may be shaped and configured in any of a variety of suitable shapes, including, by way of non-limiting example, rectangular, triangular, ovoid, linear, and non-circumferential shapes. The radiopaque markers 270 may be formed of any of a variety of biocompatible radiopaque materials that are sufficiently visible under fluoroscopy to assist in the procedure. Such radiopaque materials may be fabricated from, by way of non-limiting example, platinum, gold, silver, platinum/iridium alloy, and tungsten. The markers 270 may be attached to the catheter 200 using a variety of known methods such as adhesive bonding, lamination between two layers of polymers, or vapor deposition, for example. Various embodiments may include any number and arrangement of radiopaque markers. In some embodiments, the catheter 200 lacks radiopaque markers.

In the pictured embodiment, the distal portion 260 includes an imaging apparatus 280. The imaging apparatus 280 may comprise any type of imaging apparatus that is configured for use in intravascular imaging, including without limitation intravascular ultrasound imaging (IVUS) and optical coherence tomography (OCT). Other embodiments may lack the imaging apparatus 280.

The distal portion 260 of the catheter 210 includes a pressure sensor 300 positioned at a distal tip 290. In some embodiments, the distal tip 290 is tapered to facilitate insertion of the body 220 into a patient. In other embodiments, the distal tip 290 may be blunt, angled, or rounded. The pressure sensor 300 is embedded within the catheter wall 222 of the catheter 210. In the pictured embodiment, the pressure sensor 300 is located within the distal portion 260 and is proximal to the distal tip 290. The pressure sensor 300 will be described in further detail below in relation to FIGS. 4-6.

As mentioned above, the interface 240 is configured to connect the catheter 210 to a patient interface module or controller 310, which may include a guided user interface (GUI) 315. More specifically, in some instances the interface 240 is configured to communicatively connect at least the pressure sensor 300 of the catheter 210 to the controller 310 suitable for carrying out intravascular pressure measurement. In some instances the interface 240 is configured to communicatively connect the imaging apparatus 280 to a controller 310 suitable for carrying out intravascular imaging. The controller 310 is in communication with and performs specific user-directed control functions targeted to a specific device or component of the system 200, such as the pressure sensor 300 and/or the imaging apparatus 280.

The interface 240 may also be configured to include at least one electrical connection electrically coupled to the pressure sensor 300 via a dedicated sensor cable (not shown in FIG. 3) running through the body 220 as described in more detail below with respect to FIGS. 4 and 5. Such a configuration allows for the pressure sensor 300 to be easily energized. Such a configuration may also allow the pressure sensor 300 to transmit data via the controller 310 to data display modules such as the GUI 315 and/or a processor 320. The interface 240 may be coupled to a power source 325 via the controller 310, with the controller 310 allowing energy to be selectively directed to the pressure sensor 300 when necessary.

The controller 310 may be connected to the processor 320, which is typically an integrated circuit with power, input, and output pins capable of performing logic functions. The processor 320 may include any one or more of a microprocessor, a controller, a digital signal processor (DSP), an application specific integrated circuit (ASIC), a field-programmable gate array (FPGA), or equivalent discrete or integrated logic circuitry. In some examples, processor 320 may include multiple components, such as any combination of one or more microprocessors, one or more controllers, one or more DSPs, one or more ASICs, or one or more FPGAs, as well as other discrete or integrated logic circuitry. The functions attributed to processor 320 herein may be embodied as software, firmware, hardware or any combination thereof.

In various embodiments, the processor 320 is a targeted device controller that may be connected to a power source 325, accessory devices 340, and/or a memory 345. In such a case, the processor 320 is in communication with and performs specific control functions targeted to a specific device or component of the system 200, such as the pressure sensor 300 and/or the imaging apparatus 280, without utilizing user input from the controller 310. For example, the processor 320 may direct or program the expandable structure 300 to function for a period of time without specific user input to the controller 310. In some embodiments, the processor 320 is programmable so that it can function to simultaneously control and communicate with more than one component of the system 200, including the accessory devices 340, the memory 345, and/or the power source 325. In other embodiments, the system includes more than one processor and each processor is a special purpose controller configured to control individual components of the system.

The processor 320 may include one or more programmable processor units running programmable code instructions for implementing the pressure measurement methods described herein, among other functions. The processor 320 may be integrated within a computer and/or other types of processor-based devices suitable for a variety of intravascular applications, including, by way of non-limiting example, pressure sensing and/or intravascular imaging. The processor 320 can receive input data from the controller 310, from the imaging apparatus 280 and/or the pressure sensor 300 directly via wireless mechanisms, or from the accessory devices 340. The processor 320 may use such input data to generate control signals to control or direct the operation of the catheter 210. In some embodiments, the user can program or direct the operation of the catheter 210 and/or the accessory devices 340 from the controller 310 and/or the GUI 315. In some embodiments, the processor 320 is in direct wireless communication with the imaging apparatus 280 and/or the pressure sensor 300, and can receive data from and send commands to the imaging apparatus 280 and/or the pressure sensor 300.

The power source 325 may be a rechargeable battery, such as a lithium ion or lithium polymer battery, although other types of batteries may be employed. In other embodiments, any other type of power cell is appropriate for power source 325. The power source 325 provides power to the system 200, and more particularly to the processor 320 and the pressure sensor 300. The power source 325 may be an external supply of energy received through an electrical outlet. In some examples, sufficient power is provided through on-board batteries and/or wireless powering.

The various peripheral devices 340 may enable or improve input/output functionality of the processor 320. Such peripheral devices 340 include, but are not necessarily limited to, standard input devices (such as a mouse, joystick, keyboard, etc.), standard output devices (such as a printer, speakers, a projector, graphical display screens, etc.), a CD-ROM drive, a flash drive, a network connection, and electrical connections between the processor 320 and other components of the system 200. By way of non-limiting example, the processor 320 may manipulate data from the pressure sensor 300 to generate a pressure ratio (i.e. FFR) value, evaluate the severity of the lesion or stenosis, and may suggest an appropriate treatment for the patient based on the pressure ratio and/or the flow data. The peripheral devices 340 may also be used for downloading software containing processor instructions to enable general operation of the catheter 210, and for downloading software implemented programs to perform operations to control, for example, the operation of any auxiliary devices attached to the catheter 210. In some embodiments, the processor may include a plurality of processing units employed in a wide range of centralized or remotely distributed data processing schemes.

The memory 345 is typically a semiconductor memory such as, for example, read-only memory, a random access memory, a FRAM, or a NAND flash memory. The memory 345 interfaces with processor 320 such that the processor 320 can write to and read from the memory 345. For example, the processor 320 can be configured to read data from the pressure sensor 300, calculate pressure ratios (i.e. FFR) from that data, and write that data and the calculated ratios to the memory 345. In this manner, a series of pressure readings and/or calculated pressure ratios can be stored in the memory 345. The processor 320 is also capable of performing other basic memory functions, such as erasing or overwriting the memory 345, detecting when the memory 345 is full, and other common functions associated with managing semiconductor memory.

The controller 310 may be configured to couple the pressure sensor 300 to the processor 320. In some embodiments, under the user-directed operation of the controller 310, the processor 320 may generate a selected sequence or frequency of pressure readings best suited to a particular application. As described above, in some embodiments, at least one sensor wire (not shown in FIG. 3) passing through the body 220 and the interface 240 connects the pressure sensor 300 to the controller 310 and/or the processor 320. The user may use the controller 130 to initiate, terminate, and adjust various operational characteristics of the pressure sensor 300.

FIG. 4 illustrates the catheter 210 surrounding a guidewire 400 in an over-the-wire configuration. In an over-the-wire configuration, the catheter 210 is configured to be fully withdrawn over the guidewire 400, and the guidewire 400 may travel through the entire length of the catheter body 220. In some embodiments, the guidewire 400 travels through a discrete guidewire lumen. In other embodiments, the guidewire 400 travels through the lumen 225. The guidewire 400 retains full rotational and coaxial mobility relative to the catheter 210. In the over-the-wire configuration, the guidewire 400 is necessarily longer than the catheter 210 in order to allow a distal end 405 of the guidewire 400 to emerge from the distal end 247 of the catheter 210 and to enable the user to manipulate a proximal end (not shown) of the guidewire 400.

FIG. 4 illustrates the pressure sensor 300 in greater detail. The pressure sensor 300 is shown embedded in the catheter wall 222. The pressure sensor 300 comprises any type of pressure sensor that is sufficiently stress resistant to maintain functionality while embedded within the catheter wall 222. For example, the pressure sensor 300 may comprise a capacitive sensor, a piezoresistive pressure transducer, a fiber optic pressure sensor, a sensor with a silicon backbone (e.g., a Mercury sensor), or any other type of pressure sensor having the requisite durability and stress resistance. In some instances, the sensor 300 includes an array or plurality of sensor elements (e.g., a capacitive pressure sensor array). In the pictured embodiment, the sensor 300 includes a sensor diaphragm assembly 407. In some embodiments, the sensor diaphragm assembly 407 includes a body having a recess covered by a flexible diaphragm configured to measure fluid pressure. The diaphragm may flex in response to variations in pressure around the diaphragm, thereby reflecting variations in blood pressure, for example. The sensor 300 can then measure and transmit the variations in pressure imparted on the diaphragm assembly 407.

In the pictured embodiment, the sensor 300 is positioned within a sensor recess 410 within the catheter wall 222. In some embodiments, the sensor 300 is in intimate contact with the wall 222. The sensor 300 may be coupled to the catheter wall 222 using any of a variety of known connection methods, including by way of non-limiting example, welding, biologically-compatible adhesive, and/or mechanical fasteners. For example, in one embodiment, the sensor 300 is adhesively bonded to the sensor recess 410 using Loctite 3311 or any other biologically compatible adhesive. In some embodiments, the sensors may be integrally formed with the catheter wall 222. In some embodiments (e.g., FIG. 9), the sensor recess may be radiopaque.

A communication channel 415 extends proximally from the sensor recess 410 toward the adapter 230 (shown in FIG. 3). In some embodiments, the communication channel 415 includes at least one sensor wire 420 that transfers sensed data from the sensor 300 to the adapter 230, the controller 310, and/or the processor 320 (shown in FIG. 3). In some embodiments, the sensor wire 420 or another wire within the communication channel 415 supplies power to the sensor 300. In other embodiments, the sensor wire 420 is embedded directly into the wall 222 without a discrete communication channel 415. At least one sensor wire 420 connects each sensor 300 to the adapter 230, the controller 310, and/or the processor 320 (shown in FIG. 3). In alternate embodiments, several sensors 300 may be embedded within the wall 222 and coupled adapter 230, the controller 310, and/or the processor 320 using one or more shared sensor wires. In other embodiments, each sensor 300 may communicate with the adapter 230, the controller 310, and/or the processor 320 via wireless means

The sensor 300 is sealed within the wall 222 by a sensor cover 425. The sensor cover 425 isolates and protects the sensor 300 from the environment surrounding the catheter 210. The sensor cover 425 may be formed of any of a variety of suitable biocompatible materials, such as, by way of non-limiting example, silicone, polymer, pebax, nylon, PTFE, polyurethane, PET, and/or combinations thereof. The sensor cover 425 is shaped to lie flush with the catheter wall 222. In other words, an outer surface 430 of the catheter 210 and an exterior surface 431 of the sensor cover 425 are substantially aligned so that the outer diameter D2 of the catheter 210 remains substantially unchanged in the area of the sensor 300 compared to the remainder of the catheter 210. The outer surface 430 of the catheter 210 and/or the exterior surface 431 of the sensor cover 425 may be coated with a hydrophilic or hydrophobic coating.

Other catheter embodiments may include a variety of other sensors embedded within or associated with the wall 222. As a result, the catheter 210 may be capable of simultaneously examining a number of different characteristics of the target tissue, the surrounding environment, and/or the catheter 210 itself within the body of a patient, including, for example, vessel wall temperature, blood temperature, electrode temperature, fluorescence, luminescence, and flow rate, in addition to pressure.

FIG. 5 illustrates a discrete portion 425 of the catheter 210 including the sensor 300 (shown without the guidewire 400). In the pictured embodiment, the catheter wall 222 includes a section 222a and an opposite section 222b that cooperate to form the body 220 of the catheter 210. The sensor 300 is embedded within the section 222a. The section 222a and the section 222b may have different thicknesses T1 and T2, respectively. In particular, the section 222a containing the sensor 300 may be thicker than the section 222b. For example, in the pictured embodiment, the thickness T1 of the section 222a may range from 0.001 in. to 0.006 in., and the thickness T2 of the section 222b may range from 0.001 in. to 0.004 in. In one embodiment, the thickness T1 is 0.005 in. and the thickness T2 is 0.003 in. In other embodiments, the catheter wall 222 may have a uniform thickness.

The lumen 225 includes an internal diameter D1 that is sized and shaped to accommodate the passage of the guidewire 400. The internal diameter D1 may range from 0.014 in. to 0.035 in. In one embodiment, the internal diameter D1 is 0.016 in. In one embodiment, the internal diameter D1 is 0.024 in. In one embodiment, the internal diameter D1 is 0.014 in. In another embodiment, the internal diameter D1 is 0.035 in. The catheter 210 includes an outer diameter D2 that is sized and shaped to traverse bodily passageways. In the pictured embodiment, the outer diameter is sized to allow passage of the catheter through vascular passageways. In some instances, as mentioned above, the body 220 has an external diameter D2 ranging from 0.014 inches to 0.040 inches. In one embodiment, the outer diameter D2 is 0.024 in. In one embodiment, the outer diameter D2 is 0.018 in. In another embodiment, the outer diameter D2 is 0.035 in.

FIG. 6 illustrates a catheter 210′ associated with the guidewire 400 in a rapid exchange or monorail configuration. The catheter 210′ is substantially similar to the catheter 210 except for the differences described herein. In particular, to enable the rapid exchange configuration, the catheter 210′ includes the guidewire port 265 (as shown in FIG. 3) from which the guidewire 400 exits the catheter 210′. The guidewire port 265 is positioned a short distance away from the distal end 247′ of the catheter 210′. The rapid exchange configuration enables the user to perform a pressure sensing procedure using a relatively short guidewire because only a short portion of the guidewire extends through the catheter 210′.

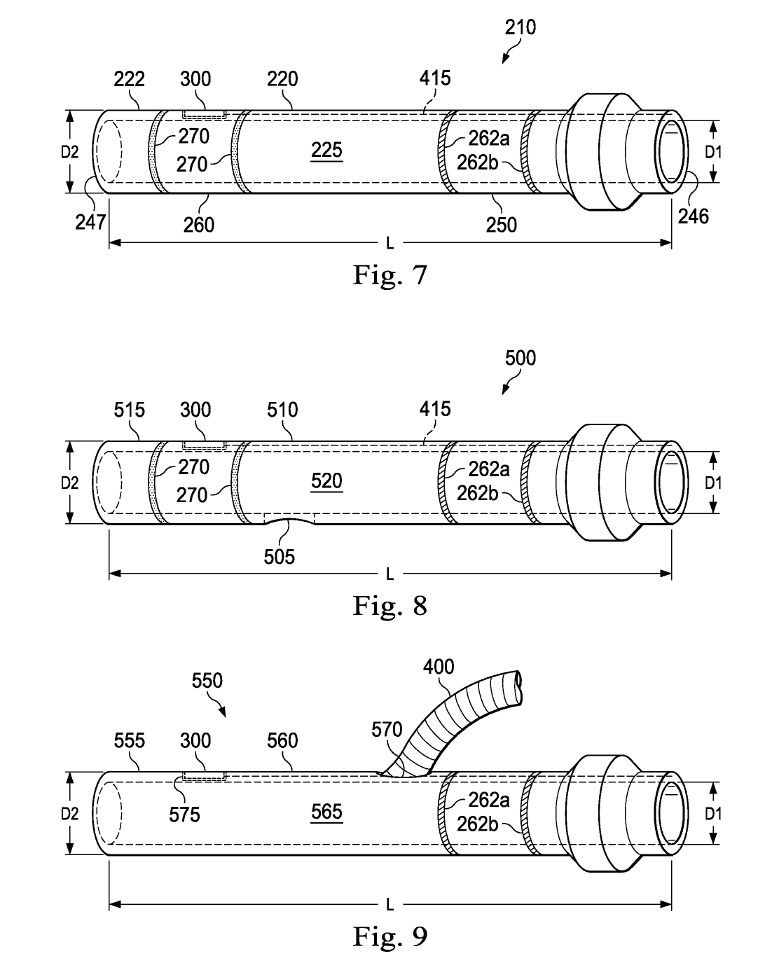

FIG. 7 illustrates another view of the catheter 210 according to one embodiment of the present disclosure. As described above, the body 220 is an elongate flexible tube that defines the lumen 225 and the longitudinal axis CA of the catheter. The wall 222 and the body 220 is configured to flex in a substantial fashion to traverse tortuous intravascular pathways. The catheter 210 may be manufactured in a variety of lengths, diameters, dimensions, and shapes. The catheter 210 includes a length L extending from the proximal end 246 to the distal end 247. In one instance, the catheter 210 has a length L of at least 90 cm and in some embodiments extending to 250 cm. In one particular embodiment, the elongated body 220 may be manufactured to have the length L of approximately 135 cm. In another embodiment, the elongated body 220 may have the length L of approximately 180 cm. Other lengths are also contemplated. In some instances, as mentioned above, the body 220 has an internal diameter D2 ranging from 0.014 inches to 0.035 inches (i.e., 0.356 mm to 0.889 mm). These examples are provided for illustrative purposes only, and are not intended to be limiting.

As shown in FIGS. 3-5, the catheter 210 includes the pressure sensor 300 embedded within the catheter wall 222. In the pictured embodiment, the sensor wire 415 is also embedded within the catheter wall 222.

In the pictured embodiment, the catheter 210 includes two radiopaque markers 270 that flank the sensor 300. Image guidance using the imaging apparatus 280 (shown in FIG. 3) or external imaging, e.g., radiographic, CT, or another suitable guidance modality, or combinations thereof, can be used to aid the user's manipulation of the catheter 210. The radiopaque markers 270 are spaced along the distal portion 260 of the catheter 210 at specific intervals from each other and at a specific distance from the distal end 247 and the sensor 300. The radiopaque markers 270 may aid the user in visualizing the path and ultimate positioning of the catheter 210 and the sensor 300 within the vasculature of the patient. In addition, the radiopaque markers 270 may provide a fixed reference point for co-registration of various imaging modalities and interventions, including by way of non-limiting example, external imaging including angiography and fluoroscopy, imaging by the imaging apparatus 280, and pressure measurement by the pressure sensor 300. Other embodiments may lack radiopaque markers.

As described above, in the pictured embodiment, the catheter 210 can include shaft markers 262 disposed along the proximal portion 250 of the catheter 210 to aid in positioning the catheter in the body of a patient. The shaft markers 262 may be positioned a specific distance from each other and comprise a measurement scale reflecting the distance of the marker 262 from the sensor 300 and/or the distal end 247. The proximal portion 250 may include any number of shaft markers 262 positioned a fixed distance away from the sensor 300 associated with a range of expected distances from the patient's skin surface at the point of catheter entry to the desired area of pressure measurement and/or other intervention. In the pictured embodiment, a shaft marker 262a is positioned approximately 10 cm from a shaft marker 262b. Shaft marker 262a is positioned approximately 90 cm from the sensor 300 to reflect a standard distance of advancement from a radial access point, and the shaft marker 262a is positioned approximately 100 cm from the sensor 300 to reflect a standard distance of advancement from a femoral access point. Additional shaft markers 262 may be marked on the catheter 210 to indicate more lengths and distances.

After initially positioning the sensor 300 within the target vessel, the user may utilize the shaft markers 262 to knowledgeably shift or reposition the catheter 210 along the intravascular target vessel to measure pressure at desired locations (e.g., relative to any lesions) and/or intervals along the target vessel before, after, or without employing imaging guidance. By noting the measurement and/or change in measured distance indicated by the shaft markers 262 located immediately external to the patient's body as the catheter 210 is shifted, the user may determine the approximate distance and axial direction the sensor 300 has shifted within the patient's vasculature. In addition, the user may use the measurement and/or change in measured distance indicated by the shaft markers located immediately external to the patient's body to cross reference the intravascular position of the pressure sensor 300 indicated by intravascular imaging. In some embodiments, the shaft markers 262 may be radiopaque or otherwise visible to imaging guidance. Other embodiments may lack shaft markers.

FIG. 8 illustrates a pressure-sensing catheter 500 in accordance with one embodiment of the present disclosure. The catheter 500 is substantially similar to the catheter 210 described above in reference to FIG. 7 except for the differences described herein (i.e., the catheter 500 includes a body 510 having a wall 515 and a lumen 520 that is substantially similar to the body 220, the wall 522, and the lumen 525, respectively, of the catheter 210). In particular, the catheter 500 includes a perfusion port 505. In some instances, the perfusion port may be the same as the perfusion port 261 described above in relation to FIG. 3. In the pictured embodiment, the perfusion port 505 forms an aperture in the wall 515 of the body 510 of the catheter 500 that allows the flow of fluid and environmental contents from the exterior of the catheter 500 into the lumen 520 of the catheter 500. In other embodiments, the perfusion port comprises a plurality of smaller apertures or a sieve-like element that allows for the passage of a similar volume of fluid into the lumen 520 as a single larger aperture. By allowing fluid to flow into the lumen 520 through the perfusion port 505 during pressure measurements, the perfusion port 505 relieves the cross-sectional diameter burden added by the presence of the catheter in the vessel. In effect, the perfusion ports may improve the accuracy in measuring the pressure drop across a lesion because the pressure drop attributable to the catheter itself would be lessened by decreasing the effective cross-sectional area of the device.

The pressure-sensing catheters described herein may include any number and arrangement of perfusion ports, and the perfusion ports may be of varied shapes and sizes. For example, in some embodiments, the catheter may include only one perfusion port such as the perfusion port 505 in catheter 500. In other embodiments, the pressure-sensing catheter may include no perfusion ports, as described above with respect to catheter 210 in FIG. 7. In other embodiments, the pressure-sensing catheter may include several perfusion ports that are arranged in a symmetrical or an asymmetrical pattern on either side of the pressure sensor 300. In addition, the perfusion ports may be arranged in a symmetrical or an asymmetrical pattern around the circumference of the catheter about the longitudinal axis CA. For example, in some embodiments, the perfusion ports may be clustered on one hemispherical side of the body of the catheter (e.g., only on one side of the catheter). In other embodiments, the perfusion ports may be arranged around the circumference of the catheter. Various possible configurations of the perfusion ports are described below in reference to FIGS. 9-14. These configurations are not limited to the particular embodiments in which they are illustrated, and may be present in any of the pressure-sensing catheters described herein.

FIG. 9 illustrates a pressure-sensing catheter 550 including the sensor 300 in accordance with one embodiment of the present disclosure. The catheter 550 is substantially similar to the catheter 210 shown in FIG. 7 except for the differences described herein. As an initial matter, the catheter 550 includes a body 555 having a wall 560 and a lumen 565 that is substantially similar to the body 220, the wall 522, and the lumen 525, respectively, of the catheter 210. However, the catheter 550 is configured as a rapid exchange catheter and the catheter 550 lacks the radiopaque markers 270. In that regard, the catheter 550 includes a guidewire port 570 from which the guidewire 400 may exit the catheter 550. The guidewire 400 may traverse the catheter 550 in a similar manner as shown with respect to the embodiment shown in FIG. 6. Instead of radiopaque markers 270, the catheter 550 includes a radiopaque sensor recess 575. The radiopaque sensor recess 575 may help the user accurately position the sensor 300 relative to a lesion, in a similar fashion as described above with respect to the radiopaque markers 270. Any of the embodiments disclosed herein may employ a similar radiopaque sensor housing, in addition to or without the radiopaque markers 270.

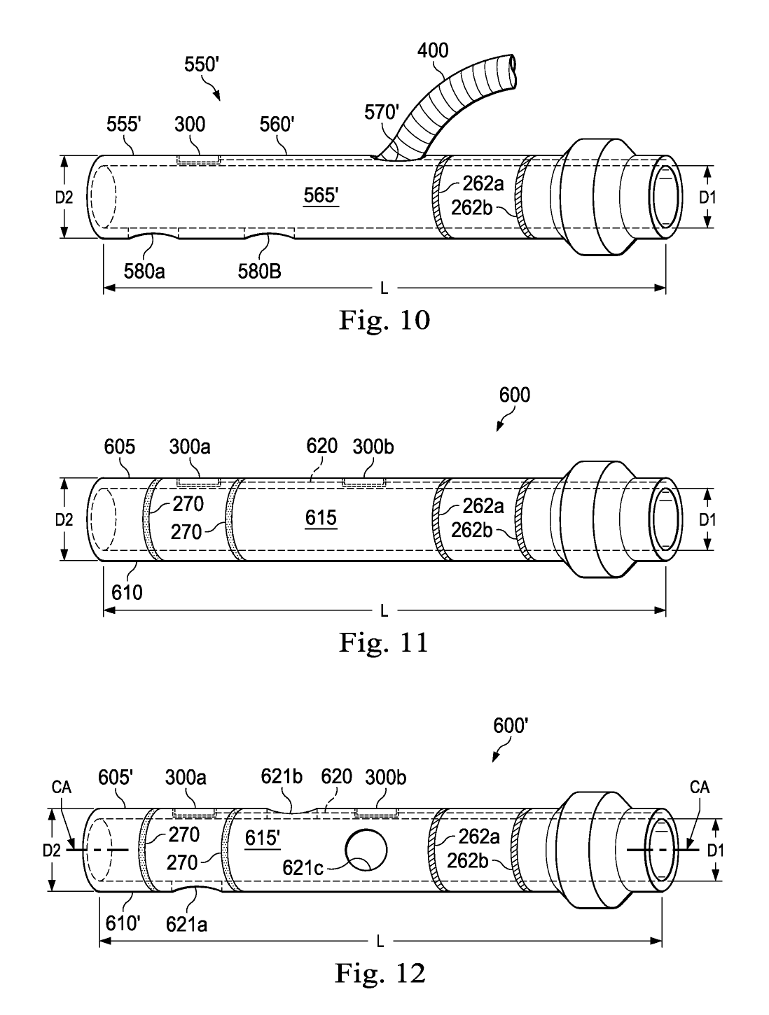

FIG. 10 illustrates a rapid exchange pressure-sensing catheter 550′ in accordance with one embodiment of the present disclosure. The catheter 550′ is substantially similar to the catheter 550 described above in reference to FIG. 9 except for the differences described herein (i.e., the catheter 550′ includes a body 555′ having a wall 560′ and a lumen 565′ that is substantially similar to the body 555, the wall 560, and the lumen 565, respectively, of the catheter 550). In particular, the catheter 550′ includes perfusion ports 580a and 580b. In some instances, the perfusion ports 580a and 580b may each be the same as the perfusion port 505 described above in relation to FIG. 8. In the pictured embodiment, the perfusion ports 580a and 580b flank the sensor 300 and form apertures in the wall 560′ of the body 555′ of the catheter 550′ that allows the flow of fluid and environmental contents from the exterior of the catheter 550′ into the lumen 565′. In other embodiments, the perfusion ports may comprise a plurality of smaller apertures or sieve-like elements that allows for the passage of a similar volume of fluid into the lumen 565′ as a single larger aperture. By allowing fluid to flow into the lumen 565′ through the perfusion ports 580a and 580b during pressure measurements, the perfusion ports relieve the cross-sectional diameter burden added by the presence of the catheter 550′ in the vessel.

FIG. 11 illustrates a pressure-sensing catheter 600 including two pressure sensors 300a and 300b. The catheter 600 is substantially similar to the catheter 210 shown in FIG. 7 except for the differences described herein. As an initial matter, the catheter 600 includes a body 605 having a wall 610 and a lumen 615 that is substantially similar to the body 220, the wall 522, and the lumen 525, respectively, of the catheter 210. However, the catheter 600 includes multiple pressure sensors 300a and 300b connected by a sensor wire 620. In some embodiments, the sensors 300a and 300b may be spaced apart sufficiently (e.g., a fixed distance apart) to span a typical stenotic lesion. The sensor wire 620 may be the same as the sensor wire 420 described above in relation to FIG. 4. In that regard, as described in further detail below with respect to FIGS. 17A and 17B, the user may position the catheter 600 within a patient such that the sensors 300a and 300b flank the lesion, thereby allowing pressure readings both proximal and distal to the lesion at the same time, without repositioning the catheter relative to the lesion. It should be noted that certain embodiments could have more than two sensors, and that the spacing between adjacent sensors can be varied.

FIG. 12 illustrates a pressure-sensing catheter 600′ in accordance with one embodiment of the present disclosure. The catheter 600′ is substantially similar to the catheter 600 described above in reference to FIG. 11 except for the differences described herein (i.e., the catheter 600′ includes a body 605′ having a wall 610′ and a lumen 615′ that are substantially similar to the body 605, the wall 610, and the lumen 615, respectively, of the catheter 600). In particular, the catheter 600′ includes multiple perfusion ports 621a, 621b, and 621c. In some instances, the perfusion ports 621a, 621b, and 621c may each be the same as the perfusion port 505 described above in relation to FIG. 8. In the pictured embodiment, the perfusion port 621a is positioned opposite the sensor 300a, the perfusion port 621b is positioned between the two sensors 300a and 300b, and the perfusion port 621c is positioned adjacent the sensor 300b. As illustrated by FIG. 12, the perfusion ports are arranged asymmetrically about the sensors 300a and 300b, and are also arranged asymmetrically about the central axis CA of the catheter 600′ (e.g., if the perfusion port 621b is viewed as positioned at the 12 o'clock position, the perfusion port 621a is positioned at the 6 o'clock position, and the perfusion port 621c is positioned at the 9 o'clock position). The perfusion ports 621a, 621b, and 621c form apertures in the wall 610′ of the catheter 600′ that allows the flow of fluid and environmental contents from the exterior of the catheter 600′ into the lumen 615′. In other embodiments, the perfusion ports may comprise a plurality of smaller apertures or sieve-like elements that allows for the passage of a similar volume of fluid into the lumen 615′ as a single larger aperture. By allowing fluid to flow into the lumen 615′ through the perfusion ports 621a, 621b, and 621c during pressure measurements, the perfusion ports relieve the cross-sectional diameter burden added by the presence of the catheter 600′ in the vessel.

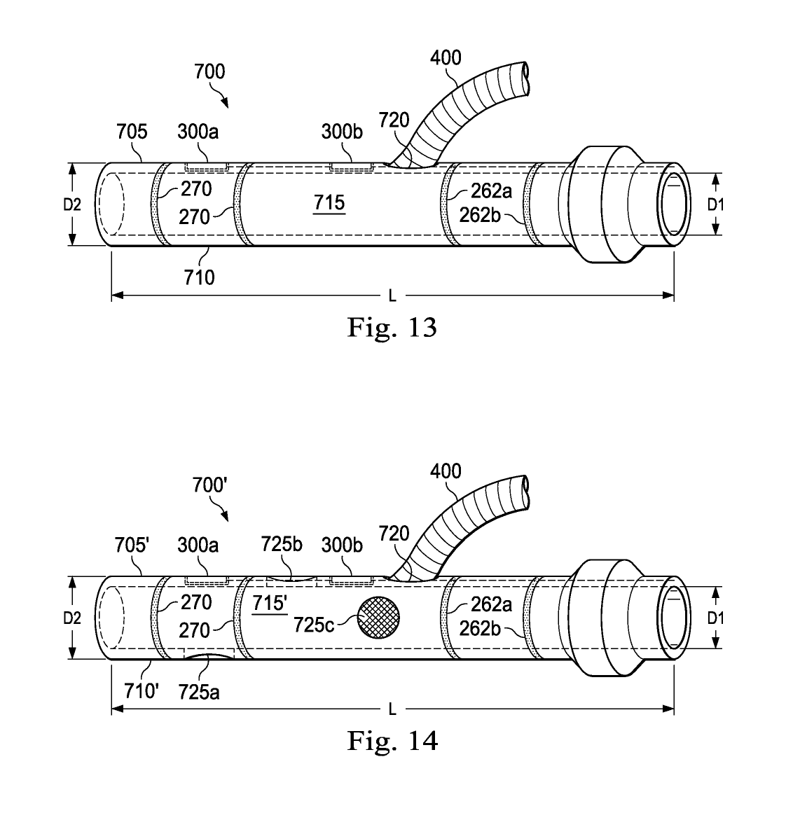

FIG. 13 illustrates a pressure-sensing catheter 700 including the sensors 300a and 300b in accordance with one embodiment of the present disclosure. The catheter 700 is substantially similar to the catheter 600 shown in FIG. 11 except for the differences described herein. As an initial matter, the catheter 700 includes a body 705 having a wall 710 and a lumen 715 that is substantially similar to the body 605, the wall 610, and the lumen 615, respectively, of the catheter 600. However, the catheter 700 is configured as a rapid exchange catheter. In that regard, the catheter 700 includes a guidewire port 720 from which the guidewire 400 may exit the catheter 700. The guidewire 400 may traverse the catheter 700 in a similar manner as shown with respect to the embodiment shown in FIG. 6.

FIG. 14 illustrates a rapid exchange pressure-sensing catheter 700′ in accordance with one embodiment of the present disclosure. The catheter 700′ is substantially similar to the catheter 700 described above in reference to FIG. 13 except for the differences described herein (i.e., the catheter 700′ includes a body 705′ having a wall 710′ and a lumen 715′ that is substantially similar to the body 705, the wall 710, and the lumen 715, respectively, of the catheter 700). In particular, the catheter 700′ includes perfusion ports 725a, 725b, and 725c. In some instances, the perfusion ports 725a, 725b, and 725c may each be similar to the perfusion port 505 described above in relation to FIG. 8. In the pictured embodiment, the perfusion port 725a is positioned opposite the sensor 300a, the perfusion port 725b is positioned between the two sensors 300a and 300b, and the perfusion port 725c is positioned adjacent the sensor 300b. As illustrated by FIG. 14, the perfusion ports are arranged asymmetrically about the sensors 300a and 300b, and are also arranged asymmetrically about the central axis CA of the catheter 600′ (e.g., if the perfusion port 725b is viewed as positioned at the 12 o'clock position, the perfusion port 725a is positioned at the 6 o'clock position, and the perfusion port 725c is positioned at the 9 o'clock position). The perfusion ports 725a and 725b form apertures in the wall 610′ of the catheter 600′ that allows the flow of fluid and environmental contents from the exterior of the catheter 600′ into the lumen 615′. The perfusion port 725c forms a plurality of smaller apertures or a sieve-like element that allows for the passage of a similar volume of fluid into the lumen 715′ as a single larger aperture. By allowing fluid to flow into the lumen 715′ through the perfusion ports 725a, 725b, and 725c during pressure measurements, the perfusion ports relieve the cross-sectional diameter burden added by the presence of the catheter 700′ in the vessel.

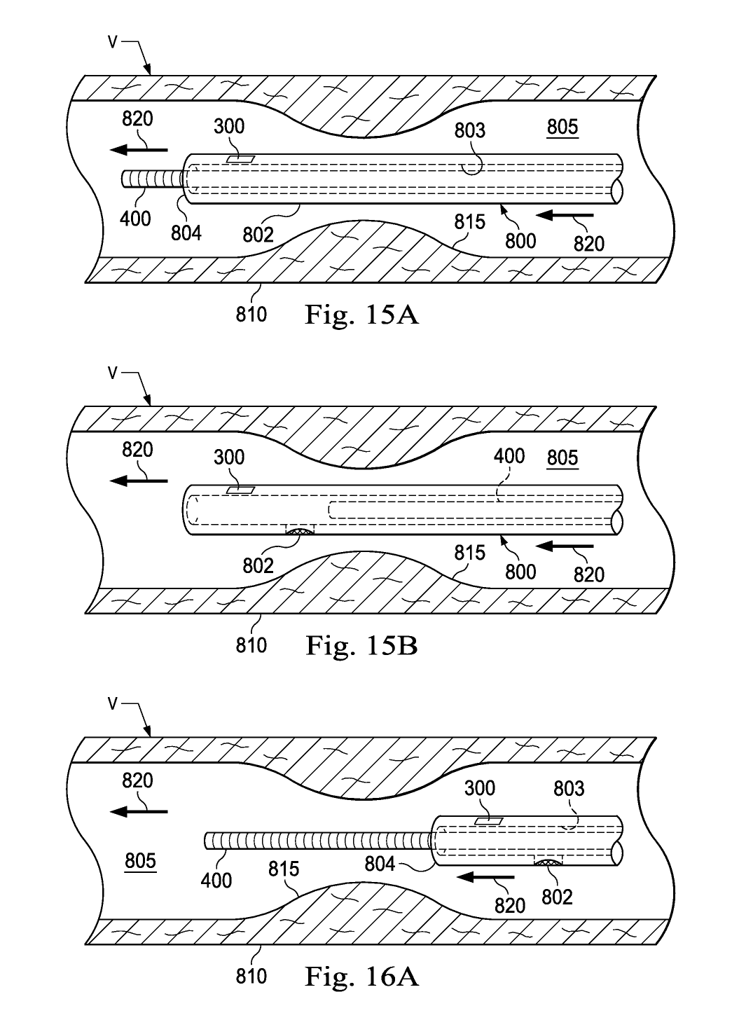

FIGS. 15A-17B illustrate methods of utilizing various pressure-sensing catheters disclosed herein to measure intravascular pressures. FIGS. 15A and 15B illustrate an exemplary pressure-sensing catheter 800 having the pressure sensor 300 positioned within a diseased vessel V. In some instances, the catheter 800 is the same as the catheter 210 shown in FIG. 3. In the pictured embodiment, the catheter 800 is configured as an over-the-wire catheter, but in other embodiments the catheter 800 may be configured as a rapid exchange catheter. In the pictured embodiment, the catheter 800 includes a perfusion port 802 and a lumen 803. The perfusion port 802 permits fluid surrounding the catheter 800 (e.g., blood) to flow through the lumen 803 of the catheter 800 (to exit the lumen 803 at a distal end 804), thereby decreasing the distorting effect the catheter 800 has on the distal pressure measurement. In particular, the perfusion port 802, by allowing circulation of fluid through the distal end of the catheter 800, decreases the overall cross-sectional blockage of the catheter 800.

The vessel V includes a lumen 805 that includes a circumferential lesion 810. The lumen 805 includes a luminal wall 815 that is irregularly shaped by the presence of the lesion 810 (e.g., an atherosclerotic plaque). Blood flows through the lumen 805 in the direction of the arrows 820. Prior to insertion of the catheter 800, the guidewire 400 may be introduced into the vasculature of a patient using standard percutaneous techniques. Once the guidewire 400 is positioned within the target blood vessel, the catheter 800 may be introduced into the vasculature of a patient over the guidewire 400 and advanced to the area of interest. In the alternative, the catheter 800 may be coupled to the guidewire 400 external to the patient and both the guidewire 460 and the catheter 800 may be introduced into the patient and advanced to an area of interest simultaneously.

The user can advance the catheter 800 over the guidewire 400 until the sensor 300 is positioned distal to or downstream of the lesion 810. The user may use radiopaque markings (e.g., radiopaque markers 270 and/or a radiopaque sensor recess 420) and/or shaft markers (e.g., shaft markers 262) on the catheter 800 to verify the desired positioning of the catheter 800 relative to the lesion. The catheter 800 may include IVUS or other imaging apparatuses 280 (as shown in FIG. 3) thereon, thereby permitting the user to precisely position the catheter 800 within the blood vessel by using in vivo, real-time intravascular imaging. Additionally or alternatively, the user may utilize external imaging, such as, by way of non-limiting example, fluoroscopy, ultrasound, CT, or MRI, to aid in the guidance and positioning of the catheter 800 within the patient's vasculature. The external and intravascular images may be co-registered to each other for side-by-side or composite display of the images.

As shown in FIG. 15B, after correct positioning is confirmed, the user can slightly retract or withdraw the guidewire 400 proximally to expose the perfusion port 802 before obtaining the distal pressure measurement. By retracting the guidewire 400 slightly and exposing the perfusion port 802, the user can increase the accuracy of the distal pressure measurement by reducing the effective obstructive profile of the catheter 800 across the stenosis. In particular, as blood flows through the perfusion port 802, the overall cross-sectional blockage created by the catheter 800 is reduced because blood is allowed to flow through at least a portion of the catheter 800 adjacent the sensor 300.

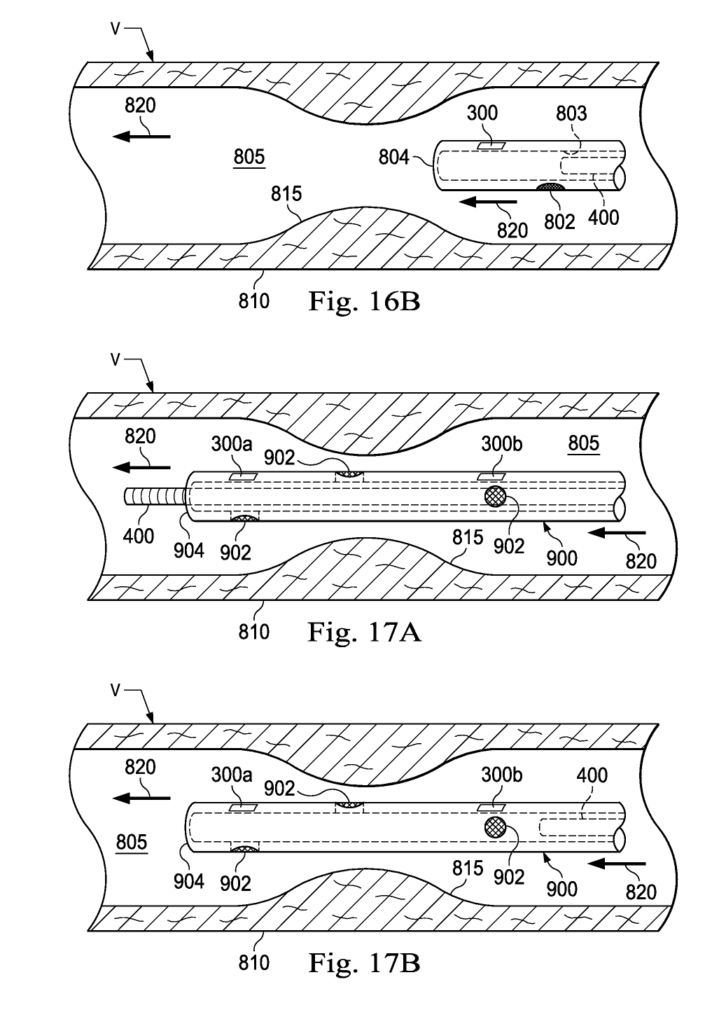

FIGS. 16A and 16B illustrate the pressure-sensing catheter 800 positioned within the diseased vessel V with the sensor 300 located proximal to or upstream of the lesion 810. As shown in FIG. 16A, after obtaining the distal pressure measurement with the sensor 300, the user can withdraw the catheter 800 over the guidewire 400 to position the sensor 300 proximal to or downstream of the lesion 810. The user may use radiopaque markings (e.g., radiopaque markers 270 and/or a radiopaque sensor recess 420) and/or shaft markers (e.g., shaft markers 262) on the catheter 800 to verify the desired positioning of the catheter 800 relative to the lesion. After correct positioning is confirmed, the user can retract or proximally withdraw the guidewire 400 to expose the perfusion port 802 again. By retracting the guidewire 400 slightly and exposing the perfusion port 802, the user can increase the accuracy of the proximal pressure measurement by reducing the effective obstructive profile of the catheter 800. Then, the user can activate the sensor 300 to obtain the proximal pressure measurement. In some instances, the user need not withdraw or retract the guidewire 400 before obtaining the proximal pressure measurement. The steps illustrated in FIGS. 15 and 16 may be repeated until all the desired pressure measurements are obtained along an area of interest in the vessel V. In addition, the steps illustrated in FIGS. 15A and 15B and FIGS. 16A and 16B may be performed in the opposite order (i.e., the proximal pressure measurement may be obtained before the distal pressure measurement). After obtaining the proximal and distal pressure measurements, the user and/or the processor 320 (shown in FIG. 3) can calculate the FFR.

FIGS. 17A and 17B illustrate an exemplary pressure-sensing catheter 900 having the pressure sensors 300a and 300b positioned within the diseased vessel V having the lesion 810. In some instances, the catheter 900 is substantially similar to the catheter 600′ shown in FIG. 12. In the pictured embodiment, the catheter 900 is configured as an over-the-wire catheter, but in other embodiments the catheter 900 may be configured as a rapid exchange catheter (e.g., similar to the catheter 700′ shown in FIG. 14). In the pictured embodiment, the catheter 900 includes multiple perfusion ports 902 and a lumen 903. The perfusion ports 902 permits fluid surrounding the catheter 900 (e.g., blood) to flow through the lumen 903 of the catheter 900 (to exit the lumen 903 at a distal end 904), thereby decreasing the distorting effect the catheter 900 has on the distal pressure measurement. In particular, the perfusion ports 902, by allowing circulation of fluid through the distal end 904 of the catheter 900, decreases the overall cross-sectional blockage of the catheter 900.

Prior to insertion of the catheter 900, the guidewire 400 may be introduced into the vasculature of a patient using standard percutaneous techniques. Once the guidewire 400 is positioned within the target blood vessel, the catheter 900 may be introduced into the vasculature of a patient over the guidewire 400 and advanced to the area of interest. In the alternative, the catheter 900 may be coupled to the guidewire 400 external to the patient and both the guidewire 460 and the catheter 900 may be introduced into the patient and advanced to an area of interest simultaneously.

The user can advance the catheter 900 over the guidewire 400 until the sensor 300a is positioned distal to or downstream of the lesion 810 and the sensor 300b is positioned proximal to or upstream of the lesion 810. The user may use radiopaque markings (e.g., radiopaque markers 270 and/or a radiopaque sensor recess 420) and/or shaft markers (e.g., shaft markers 262) on the catheter 900 to verify the desired positioning of the catheter 900 relative to the lesion. The catheter 900 may include IVUS or other imaging apparatuses 280 (as shown in FIG. 3) thereon, thereby permitting the user to precisely position the catheter 900 within the blood vessel by using in vivo, real-time intravascular imaging. Additionally or alternatively, the user may utilize external imaging, such as, by way of non-limiting example, fluoroscopy, ultrasound, CT, or MRI, to aid in the guidance and positioning of the catheter 900 within the patient's vasculature. The external and intravascular images may be co-registered to each other for side-by-side or composite display of the images.

As shown in FIG. 17B, after correct positioning is confirmed, the user can slightly retract or withdraw the guidewire 400 proximally to expose the perfusion ports 902 before obtaining the pressure measurements. By retracting the guidewire 400 slightly and exposing the perfusion ports 902, the user can increase the accuracy of the distal pressure measurement by reducing the effective obstructive profile of the catheter 900 across the stenosis. In particular, as blood flows through the perfusion port 902, the overall cross-sectional blockage created by the catheter 900 is reduced because blood is allowed to flow through at least a portion of the catheter 900 adjacent the sensors 300a and 300b. Although FIG. 17B illustrates the guidewire 400 retracted proximal to all the perfusion ports 902, in some instances, the user need only retract the guidewire proximal to the perfusion ports adjacent to or distal to the lesion 810. By retracting the guidewire 400 slightly and exposing the perfusion ports 902, the user can increase the accuracy of the pressure measurements by reducing the effective obstructive profile of the catheter 900. After exposing the perfusion ports 902, the user can activate the sensors 300a and 300b to obtain the proximal and the distal pressure measurements, respectively. The steps illustrated in FIGS. 17A and 17B may be repeated until all the desired pressure measurements are obtained along an area of interest in the vessel V. After obtaining the proximal and distal pressure measurements, the user and/or the processor 320 (shown in FIG. 3) can calculate the FFR.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example, the pressure-sensing catheters disclosed herein may be utilized anywhere with a patient's body, including both arterial and venous vessels, having an indication for pressure measurement. It is understood that such variations may be made to the foregoing without departing from the scope of the present disclosure. Accordingly, it is appropriate that the appended claims be construed broadly and in a manner consistent with the present disclosure.

We claim:

1. An apparatus for intravascular pressure measurement, comprising:
an elongate body including a proximal portion and a distal portion, the body defining a lumen extending from a proximal end to a distal end of the body, the lumen sized and shaped to allow the passage of a guidewire there through, the body including an annular wall extending from the lumen to an outer surface of the body; and
a first pressure sensor disposed within the wall of the distal portion of the body, the pressure sensor including a sensor cover coupled to the wall,
wherein an exterior surface of the sensor cover and the outer surface of the body are substantially aligned.

2. The apparatus of claim 1, further including at least one perfusion port in the wall that enables fluid communication between the lumen and environmental contents outside the elongate body.

3. The apparatus of claim 2, wherein the at least one perfusion port comprises an aperture extending through the wall from the outer surface of the body to the lumen.

4. The apparatus of claim 2, wherein the at least one perfusion port comprises a sieve-like element.

5. The apparatus of claim 2, wherein the at least one perfusion port comprises an array of apertures.

6. The apparatus of claim 1, wherein the first pressure sensor comprises a capacitive pressure sensor.

7. The apparatus of claim 1, wherein the sensor cover is formed of silicone.

8. The apparatus of claim 1, further including a second pressure sensor positioned a fixed distance from the first pressure sensor within the wall of the distal portion of the body.

9. The apparatus of claim 1, further including a guidewire port in the distal portion of the body for use in a rapid exchange configuration.

10. The apparatus of claim 1, wherein the pressure sensor is disposed within a sensor recess formed within the wall.

11. The apparatus of claim 10, wherein the sensor recess is radiopaque.

12. The apparatus of claim 1, further including at least one radiopaque marker coupled to the wall adjacent to the first pressure sensor.

13. The apparatus of claim 1, further including at least one shaft market disposed on the proximal portion of the body.

14. A method for intravascular pressure measurement within a lumen of a vessel including a lesion, comprising:
positioning a guidewire within the lumen of the vessel distal to the lesion;
advancing a pressure-sensing catheter including a first pressure sensor and at least one perfusion port over the guidewire within the lumen of the vessel such that the first pressure sensor is positioned distal to the lesion;
withdrawing the guidewire in a proximal direction until the guidewire is positioned proximal of the at least one perfusion port; and
obtaining a distal pressure measurement from the first pressure sensor.

15. The method of claim 14, further including imaging the pressure-sensing catheter to obtain image data reflecting the location of the first pressure sensor within the lumen relative to the lesion.

16. The method of claim 15, further including repositioning the pressure-sensing catheter in an optimal intravascular location for pressure measurement based on the image data.

17. The method of claim 14, further including withdrawing the pressure-sensing catheter in a proximal direction to position the first pressure sensor proximal to the lesion, withdrawing the guidewire in a proximal direction until the guidewire is positioned proximal of both the lesion and the at least one perfusion port, and obtaining a proximal pressure measurement from the first pressure sensor.

18. The method of claim 14, wherein the pressure-sensing catheter includes a second pressure sensor spaced apart from the first pressure sensor, and further including obtaining a proximal pressure measurement from the second pressure sensor without repositioning the pressure-sensing catheter.
